(19) United States
(12) Patent Application Publication
(10) Pub. No.: US 2025/0040957 A1
(43) Pub. Date: Feb. 6, 2025

(54) ATHERECTOMY CATHETER WITH SHAPED DISTAL TIP

(71) Applicant: Avinger, Inc., Redwood City, CA (US)

(72) Inventors: Himanshu N. PATEL, San Jose, CA (US); Anthony J. FERNANDEZ, San Mateo, CA (US); Vincent YEH, Fremont, CA (US)

(21) Appl. No.: 18/921,958

(22) Filed: Oct. 21, 2024

Related U.S. Application Data
(63) Continuation of application No. 17/455,655, filed on Nov. 18, 2021, now Pat. No. 12,161,360.
Application No. 17/455,655 is a continuation of application No. 16/310,470, filed on Dec. 17, 2018, now Pat. No. 11,224,459.
(60) Provisional application No. 62/357,173, filed on Jun. 30, 2016.

(51) Int. Cl. A61B 17/3207; A61B 17/00; A61D 1/02; A61M 25/00; A61M 25/01

(52) U.S. Cl. CPC A61B 17/320783; A61D 1/02; A61B 2017/00309; A61B 2017/00331; A61B 2017/00738; A61B 2017/320791; A61M 25/0054; A61M 25/0138

(57) ABSTRACT

An atherectomy device including a catheter body, a nosecone and a driveshaft. The catheter body may include a fixed jog section having a fixed curvature, and a flexible section having a greater lateral flexibility than the fixed jog section. The flexible section may include circumferential slits that are configured to allow the flexible section to passively bend in multiple directions. The nosecone may be attached to a distal end of the catheter body. The driveshaft may be configured to rotate within the catheter body and to move distally and proximally within the catheter body. A distal end of the driveshaft may include an annular cutter.

### CROSS REFERENCE TO RELATED APPLICATIONS

This application is a continuation of U.S. patent application Ser. No. 17/455,655, filed on Nov. 18, 2021, titled “ATHERECTOMY CATHETER WITH SHAPED DISTAL TIP,” now U.S. Patent Application Publication No. US-2022-0071656-A1, which is a continuation of U.S. patent application Ser. No. 16/310,470, filed on Dec. 17, 2018, titled “ATHERECTOMY CATHETER WITH SHAPEABLE DISTAL TIP,” now U.S. Pat. No. 11,224,459, which is a U.S. National Phase Application Under 35 U.S.C. § 371 of International Application No. PCT/US2017/040431, filed on Jun. 30, 2017, titled “ATHERECTOMY CATHETER WITH SHAPEABLE DISTAL TIP,” now International Publication No. WO 2018/006041, which claims priority to U.S. Provisional Patent Application No. 62/357,173, filed Jun. 30, 2016, titled “ATHERECTOMY CATHETER WITH SHAPEABLE DISTAL TIP,” each of which is herein incorporated by reference in its entirety.

This application may also be related to U.S. Publication No. 20150141816, titled “ATHERECTOMY CATHETER WITH IMAGING,” filed on Mar. 15, 2013, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are devices for treatment of an occluded body lumen, such as for the removal of occlusive materials from blood vessels. In particular, described herein are atherectomy catheters that are adapted to easily maneuver against tissue and plaque buildup within vessels for debulking.

### BACKGROUND

Atherosclerosis is disease in which accumulation of atheromatous materials builds up inside a person's arteries. Atherosclerosis occurs as part of the natural aging process, but may also occur due to a person's diet, hypertension, vascular injury, heredity, and so forth. Atherosclerosis can affect any artery in the body, including arteries in the heart, brain, arms, legs, pelvis, and kidneys. Atherosclerosis deposits may vary in their properties as well. Some deposits are relatively soft, other types may be fibrous, some are calcified, or a combination of all three. Based on the location of the plaque accumulation, different diseases may develop. For example, coronary heart disease occurs when plaque builds up in the coronary arteries, which supply oxygenated blood to the heart. If plaque buildup blocks the carotid artery, arteries located on each side of the neck that supply oxygen to the brain, a stroke may be the result.

Atherosclerosis may be treated in a number of ways including medication, bypass surgery, and catheter-based approaches. Atherectomy procedures involve excising or dislodging materials that block a blood vessel. Many atherectomy catheters typically have a substantially straight central axis. However, atherectomy catheters having a straight profile may be difficult to maneuver close enough to the inner surface of the arterial walls to remove all plaque buildup. Moreover, plaque removal can be complicated with such straight profile catheters when plaque formations accumulate in the curves and more tortuous portions of an artery.

The atherectomy catheters described herein address some of these challenges.

### SUMMARY OF THE DISCLOSURE

In general, in one embodiment, an atherectomy catheter for use in a vessel includes an elongate catheter body and an annular cutter. The elongate catheter body includes a fixed jog section with a pre-set curvature and a flexible section that has a greater flexibility than a remainder of the elongate catheter body. The fixed jog section and flexible section are formed of a frame including a plurality of circumferential slits therein.

This and other embodiments can include one or more of the following features. The frame in the fixed jog section can further include a longitudinal spine extending therethrough that does not have slits. The atherectomy catheter can further include a cutting window through which the annular cutter extends. The cutting window can be positioned distal of the fixed jog section and the flexible section so as to urge the cutter into the vessel. The atherectomy catheter can further include at least one laminating layer positioned over or under the frame of the fixed jog section. The laminating layer can be made of a polymer. The frame can be made of metal. The plurality of circumferential slits can be arranged in a repeating pattern. The fixed jog section can form an angle of 130° to 160° in the elongate catheter body. The frame can further include an annular spine without slits that extends between the fixed jog section and the flexible section. The flexible section can be configured to passively bend to angles of 130°-160°.

In general, in one embodiment, an atherectomy catheter for use in a vessel includes an elongate catheter body, an annular cutter, and an s-shaped curved portion in the elongate catheter body. The curved portion includes a frame having a plurality of annular spines connected together by a longitudinal proximal spine and a longitudinal distal spine. The longitudinal proximal spine is positioned approximately 180 degrees away from the longitudinal distal spine.

This and other embodiments can include one or more of the following features. The plurality of annular spines can include a first annular spine, a second annular spine, and a third annular spine. The longitudinal proximal spine can connect the first annular spine and the second annular spine, and the longitudinal distal spine can connect the second annular spine and the third annular spine. The atherectomy catheter can further include a cutting window through which the annular cutter extends. The cutting window can be positioned distal of the curved portion and on an outer circumference of the s-shaped curve so as to urge the cutter into the vessel. The s-shaped curved portion can be configured to be activated by pulling or pushing on a shaft of the atherectomy catheter. The atherectomy catheter can further include at least one laminating layer positioned over or under the frame. The laminating layer can be made of a polymer. The frame can be made of metal. The distal longitudinal spine can be positioned adjacent to an exposed portion of the cutter. The distal longitudinal spine can be on a same side of the elongate catheter body as the exposed portion of the cutter. The longitudinal proximal spine can form a first angle, and the longitudinal distal spine can form a second angle. The first and second angles can extend in opposite directions, and the first angle can be between 140 and 160 degrees and the second angle can be between 140 and 160 degrees. A distal-most spine of the plurality of spines can include a beveled distal edge. The atherectomy catheter can further include a nosecone configured to pivot away from the elongate body to expose the cutter. The bevel can be configured to provide space for the nosecone to pivot.

In general, in one embodiment, an atherectomy catheter for use in a vessel includes an elongate catheter body, an annular cutter, and an s-shaped curved portion in the elongate catheter body. The curved portion includes a frame having a proximal section and a distal section. The proximal section has a plurality of circumferential proximal slits and a longitudinal proximal spine without slits, and the distal section having a plurality of circumferential distal slits and a longitudinal distal spine without slits. The longitudinal proximal spine is positioned approximately 180 degrees away from the longitudinal distal spine.

This and other embodiments can include one or more of the following features. The atherectomy catheter can further include a cutting window through which the annular cutter extends. The cutting window can be positioned distal of the distal section and on an outer circumference of the s-shaped curve so as to urge the cutter into the vessel. The s-shaped curved portion can be configured to be activated by pulling or pushing on a shaft of the atherectomy catheter. The atherectomy catheter can further include at least one laminating layer positioned over or under the frame. The laminating layer can be made of a polymer. The frame can be made of metal. The plurality of circumferential proximal slits can be arranged in a first repeating pattern, and the plurality of circumferential distal slits can be arranged in a second repeating pattern. The first repeating pattern and the second repeating pattern can be circumferentially offset from one another. The distal longitudinal spine can be positioned adjacent to an exposed portion of the cutter. The distal longitudinal spine can be on a same side of the elongate catheter body as the exposed portion of the cutter. The proximal section can form a first angle, and the distal section forms a second angle. The first and second angles can extend in opposite directions, and the first angle can be between 140 and 160 degrees and the second angle can be between 140 and 160 degrees. The frame can further include an annular spine without slits extending between the proximal section and the distal section.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an atherectomy catheter having a fixed jog.

FIG. 1B shows the atherectomy catheter of FIG. 1A in a vessel.

FIG. 2 shows a drawing of the atherectomy catheter of FIG. 1A with relative angles and dimensions.

FIG. 3A shows a variation of a distal end of an atherectomy catheter that includes a user-activated curved portion with stiffening members that cause the catheter to deform to a predetermined curved configuration when activated.

FIG. 3B is a schematic showing the stiffening members of the atherectomy catheter of FIG. 3A.

FIGS. 4A and 4B show another embodiment of an atherectomy catheter with a distal curved portion.

FIG. 5A is a top view of a user-activated curved portion of an atherectomy catheter.

FIGS. 5B and 5C are perspective views of the curved portion of FIG. 5A.

FIG. 5D is a flattened view of the curved portion of FIG. 5A.

FIG. 6A shows an atherectomy catheter including another embodiment of a user-activated curved portion.

FIG. 6B shows the frame of the curved portion of FIG. 6A including annular and longitudinal spines.

FIG. 6C shows a side view of the spine of FIG. 6B.

FIG. 6D shows a cross-sectional view of the spine of FIG. 6B.

FIG. 6E is a flattened view of the spine of FIG. 6B.

FIG. 7A shows a portion of an atherectomy catheter including a fixed jog section and a flexible section.

FIG. 7B shows a flattened view of the curved portion of the catheter of FIG. 7A.

FIG. 8A shows an exemplary flexible nosecone for use with an atherectomy catheter.

FIG. 8B shows a flattened view of a portion of the nosecone of FIG. 8A.

### DETAILED DESCRIPTION

Described herein is an atherectomy catheter having an elongate body with a curved distal portion, a nosecone and a rotatable annular cutter. The curved portion (which can otherwise be called a bent/bendable portion or jog mechanism) can advantageously be used to push the cutter up against the vessel wall to enhance the efficiency of cutting.

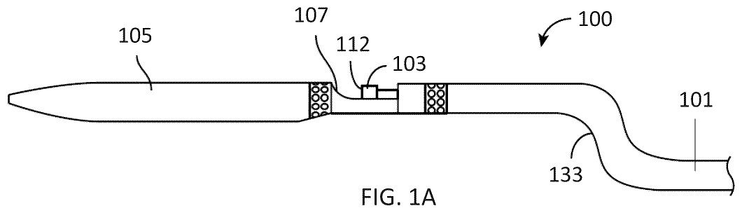

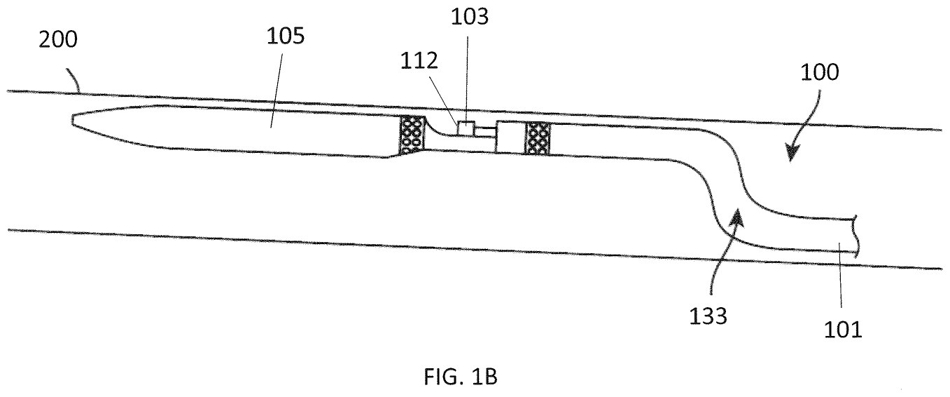

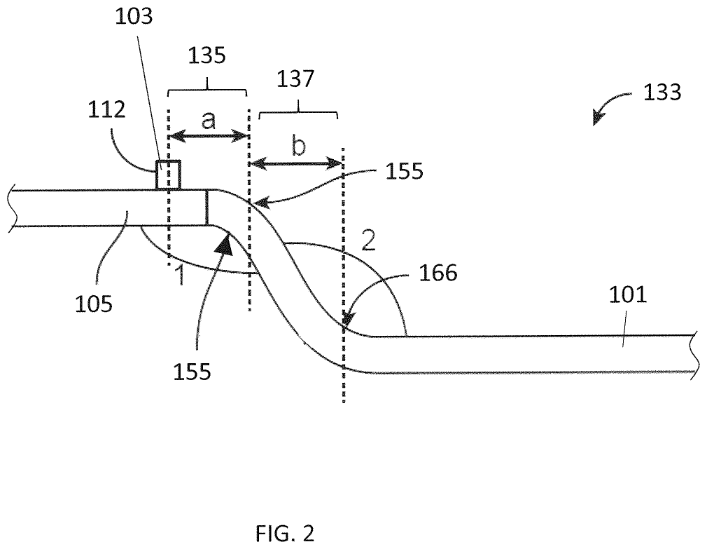

FIGS. 1A and 1B show an exemplary atherectomy catheter 100 having a curved portion along the elongate catheter body. Referring to FIGS. 1A-2, the atherectomy catheter 100 can include a catheter body 101 with a curved portion 133, a rotatable annular cutter 103 at a distal end of the catheter body 101, and a nosecone 105 at a distal end of the catheter body 101. The nosecone 105 can include a cutting window 107 configured to allow the cutter 103 to cut therethrough. The catheter 101 can further include a curved portion 133 in the catheter body 101 to radially push the cutter 103 against the vessel wall.

The curved portion 133 can be a fixed jog (i.e., have a pre-set shape). Further, the curved portion can be curved or bent such that the cutting window 107 is on the radially outermost portion of the curved portion 133 (thereby allowing the cutting window 107 to be urged against a vessel wall in use). In one embodiment, the curved portion 133 can be pre-formed, for example, by using pre-deflected shaped-set nitinol ribbon segments embedded in the outer shaft. The curved portion 133 can have two inflection points 155, 166 of opposite curvature (i.e., one curving up and the other curving down) so as to form an approximate “s” shape. In one embodiment, the s-shape can be configured such that a distal end of the catheter body 101 is offset from, but substantially parallel to, a proximal end of the catheter body 101. In other embodiments, the distal end and proximal ends of the catheter body 101 can be at a slight angle to one another so as to control the angle of cutter engagement with the vessel wall.

Thus, as shown in FIG. 2, the “s-shape” of the curved portion 133 can include a proximal section 137 have a length b that extends from the center of the distal inflection point 155 to the center of the proximal inflection point 166. Further, the curved portion 133 can include a distal section 135 having a length a that extends from the cutting edge 112 to the center of the distal inflection point 155. Further, there can be distal angle/at the distal end of the “s-shape” and a proximal angle 2 at the proximal end of the “s-shape.” These lengths (a, b) and angles (1, 2) can be tuned to achieve the desired jog or offset in order to obtain optimum apposition to tissue walls. For example, the length a can be shorter than the length b to ensure that the cutter is as close to the angle 1 as possible, thereby providing better apposition of the cutter 303. The angles 1 and 2 can be between 120 and 180 degrees, such as between 140 and 160 degrees. In one example, the length a is between 5 and 10 mm, the length b is between 10and 15 mm, the angle 1 is 140 degrees and angle 2 is 160 degrees for a catheter configured to be used in a vessel having a 2.5-4 mm diameter.

The curved portion 133 can advantageously radially push the distal end of the catheter against a vessel wall 200, thereby enabling optimized cutting and/or imaging of the vessel as shown in FIG. 1B.

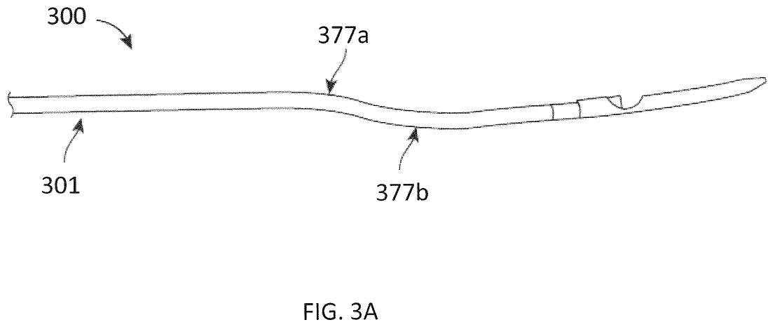

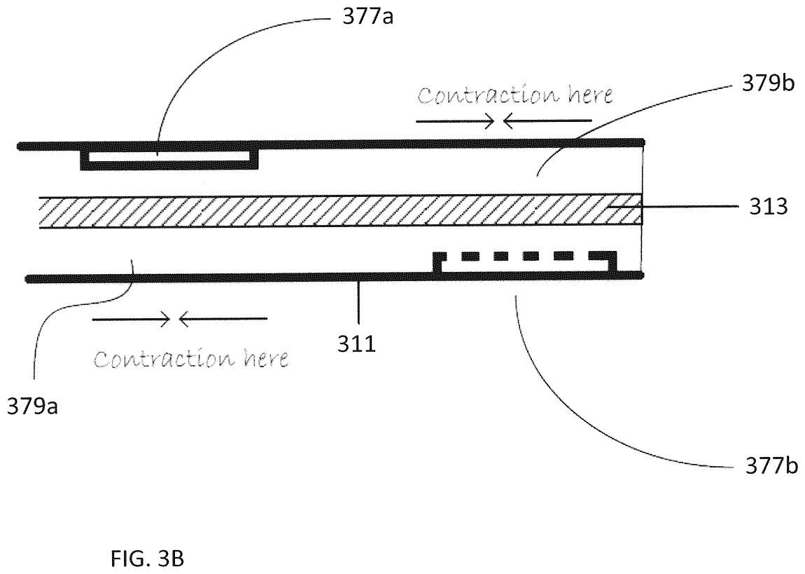

FIGS. 3A-3B show another embodiment of an exemplary catheter 300 that includes a curved portion 333 in the catheter body that urges the atherectomy cutter against the vessel wall. The curved portion 333 can have similar dimensions and features as curved portion 133. In contrast to the fixed jog curved portion 133 of catheter 100, however, the curved portion 333 can be a user-activated jog. Thus, referring to FIGS. 3A and 3B, the catheter 300, can be deflected into a curved portion 333 by tensile and compressive interaction between an inner shaft 313 (which can be a drive shaft for a cutter) and outer shaft 311 that are fixed together at the distal end but free to move relative to one another at the proximal end. The outer shaft 311 can include stiffening members 377a,b, such as nitinol or stainless steel, stiffening members, configured to bias the deflection to a set shape. As shown in FIG. 3B, there can be two stiffening members 377a, 377b that can be axially aligned with the outer shaft 311 and axially and radially offset from one another. As a result, when compression is applied on the outer shaft 311 (such as by pulling on the inner shaft 313 or a separate pullwire or shaft), the portions 379a,b of the outer shaft opposite to the stiffening members 377a,b will contract. The contraction of the two portions 379a, 379b will result in an s-shape similar to the catheter 100 shown in FIG. 1. As a result, the catheter will deflect into jog or s-shaped configuration where the distal end of the shaft is offset and parallel to the main shaft body. It is to be understood that other numbers and arrangements of stiffening members are possible, as are other resulting jog shapes.

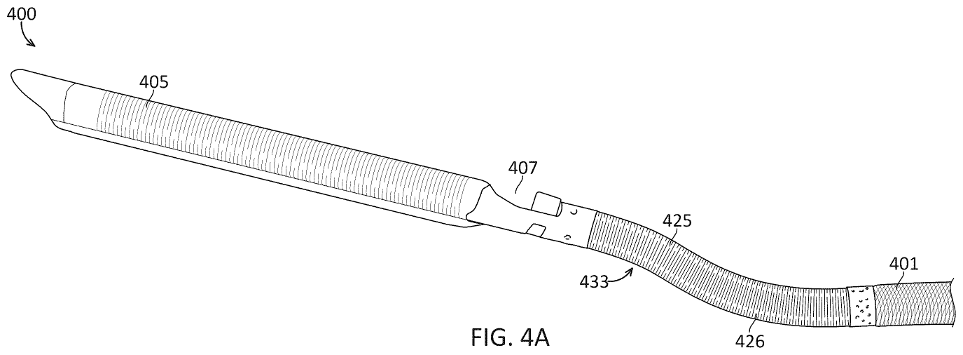

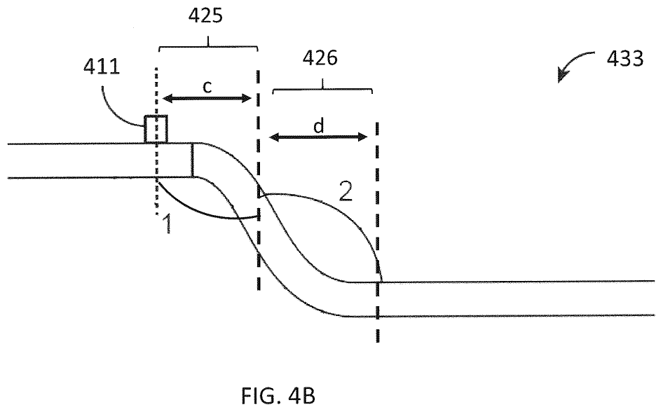

Another embodiment of an atherectomy catheter 400 including a user-activated curved portion 433 is shown in FIGS. 4A-4B. The atherectomy catheter 400 includes an elongate body 401, a nosecone 405 attached thereto, and a cutting window 407 configured to expose an annular cutter 411 therethrough. Moreover, the catheter 400 includes a curved portion 433. The curved portion 433 includes curved sections 425, 426 of opposite curvatures (i.e., one curving up and the other curving down) so as to form an approximate s-shape. In one embodiment, the s-shape can be configured such that the distal end of the catheter body 401 and/or the nosecone 405 is offset from, but substantially parallel to, a proximal end of the catheter body 401. In another embodiment, the distal end of the elongate body 401 and/or the nosecone 405 forms an angle relative to a proximal end of the catheter body 401.

Thus, as shown in FIG. 4B, the “s-shape” of the jog 433 can have a proximal curved section 426 and a distal curved section 425 having a length c. Further, there can be distal angle 1 at the distal end of the “s-shape” and a proximal angle 2 at the proximal end of the “s-shape.” The lengths (c, d) and angles (1, 2) of the jog 433 can be tuned to achieve the desired jog or offset in order to obtain optimum apposition to tissue walls. For example, the angles 1 and 2 can be between 120 and 175 degrees, such as between 140 and 160 degrees. Further, in some embodiments, the length d of the proximal section 426 is greater than the length c of the distal section 425. In one example, the length c is 5 mm, the length d is 8 mm, and the angles 1 and 2 are 150 degrees for a catheter configured to be used in a vessel having a 2.5-4 mm diameter. The curved portion 433 can be a configured to adopt the s-shape during use of the catheter, as described above with respect to curved portion 333.

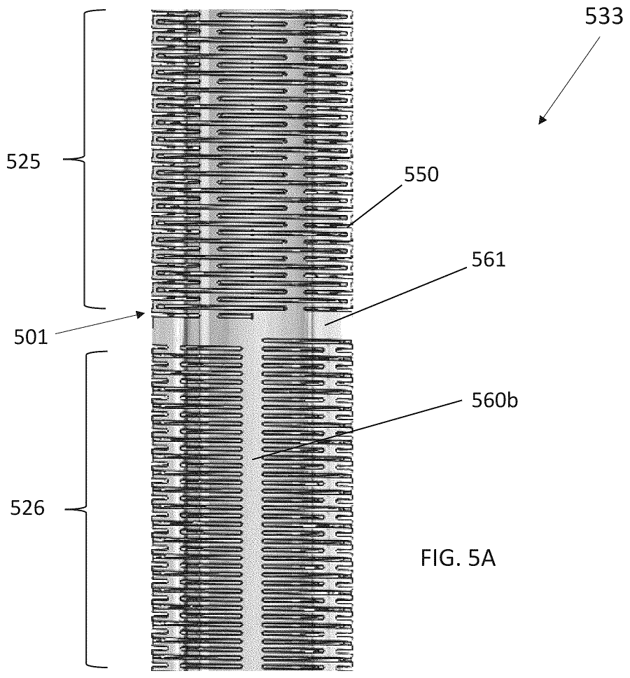

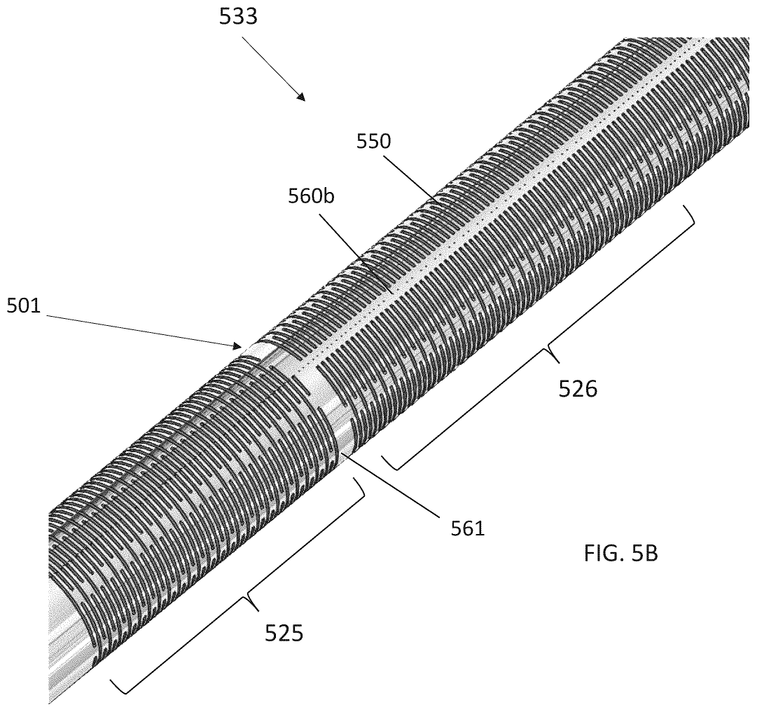

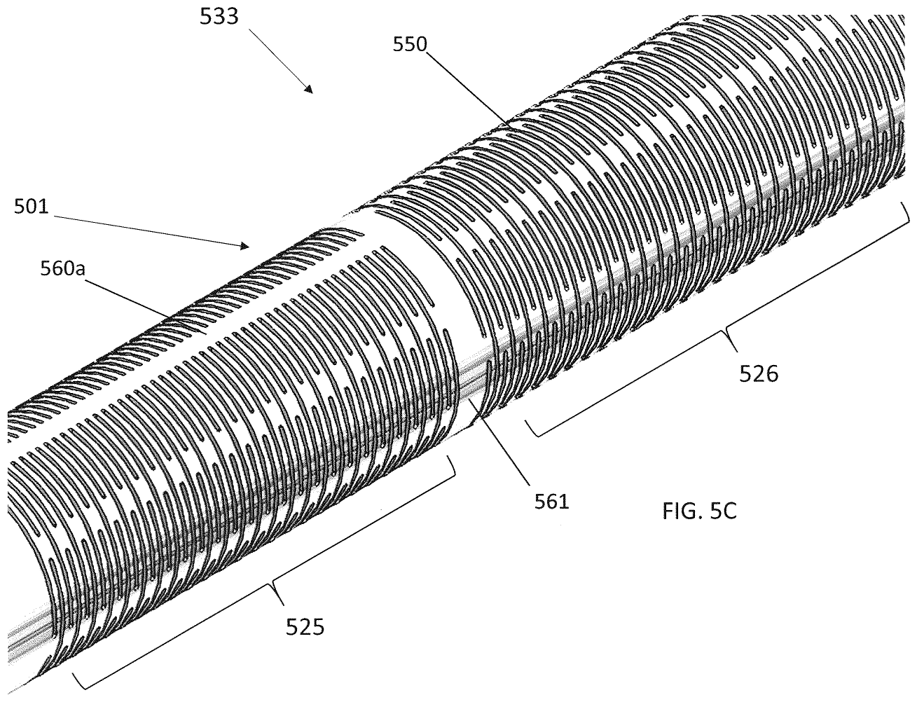

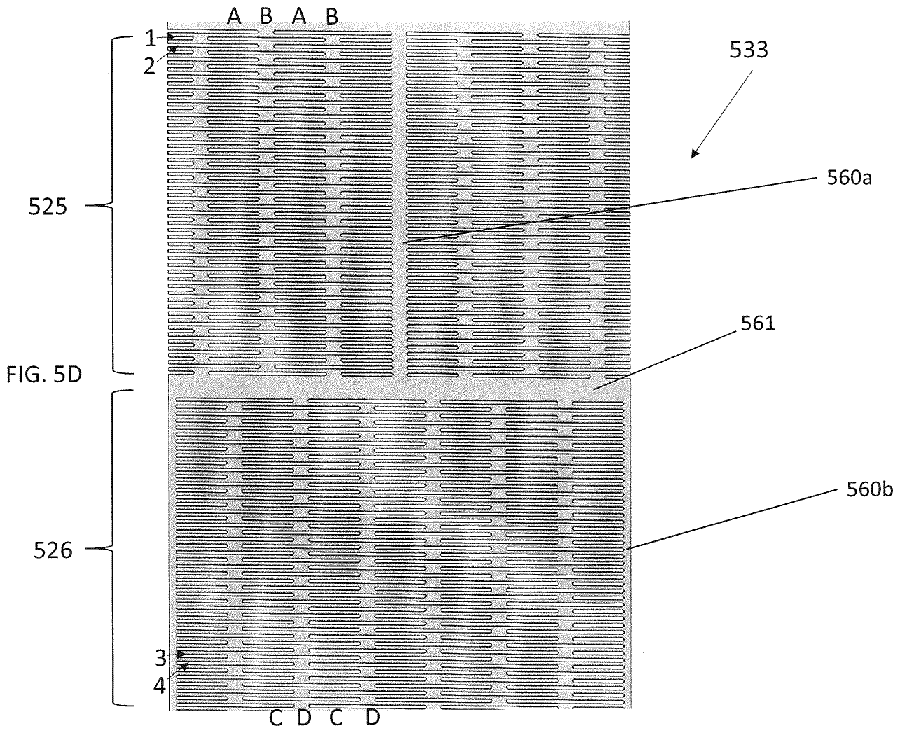

An exemplary user-activated curved portion 533 (e.g., for use as curved portion 433) is shown in FIGS. 5A-5D. The curved portion 533 can include a frame (e.g., made of Nitinol or stainless steel) including a series of circumferential slits 550 (e.g., laser cuts) that are patterned along the circumference of the elongate body 501 within the curved sections 525, 526. The frame of the curved sections 525, 526 can also include a longitudinal spine 560a,b extending therethrough. The longitudinal spines 560a,b can be positioned approximately 180 degrees away from one another (i.e., on opposites sides of the elongate body 501) and extend substantially parallel to the longitudinal central axis of the elongate body 501. The frame can further include a circumferential spine 561 separating the two curved sections 525, 526. Each spine 560a,b and 561 is formed of a substantially solid piece of material that does not include slits therein. In use, as the circumferential slits 550 compress and/or overlap with one another during bending, the longitudinal spines 560a,b form the backbone of the curved sections 525, 526. Further, in some embodiments, the frame can be laminated with a layer thereover and/or under, such as a thin polymer layer, such as Tecothane. In other embodiments, the frame is not laminated to provide for greater flexibility.

Referring to FIG. 5D, the slits 550 can be arranged in a pattern that is configured to provide flexibility while maintaining structural integrity of the elongate body. Thus, the majority of the slits 550 can have the same length, but be offset from one another. For example, the slits in distal section 525 can be arranged in rows (1,2) and columns (A, B). Each slit 550 (except the shorter slits bordering the spine 560a) can have a length equivalent to the width of columns A+B+A. Further, the slits can be offset from one another by a distance of A+B. Thus, each column A can include slits from every row 1,2 while column B can include alternating slits (from either row 1 or 2). Column B thus provides structural integrity to the slitted portion of the device. The slits in section 526 can be similarly arranged, but can be offset such that each column C (with slits from every row 3,4) is aligned with the central axis of each column D (with slits from row 3 or 4). The offset helps provide stability to the catheter as it bends.

In some examples, pushing or pulling on a shaft of the catheter, such as the cutter drive shaft, a pullshaft, or a pullwire can activate the curved portion 533. That is, as the shaft is pulled back proximally, it can place compression on the outer elongate body 501, causing the slits 550 to compress and/or move over one another while the spines 560a,b maintain their length. The resulting s-shape (see FIG. 4B) allows the cutter (just distal to spine 460a) to be pushed up against the vessel wall.

The slits 550 shown in FIGS. 5A-5D are of a repeated, symmetrical pattern. However, the pattern need not be symmetrical. In some embodiments, the slits can all have the same length. In other embodiments, some of the slits are longer than others. In one embodiments, the slits are 0.0016″ wide and 0.0575″ long with a 0.0035″ offset from the next row of slits.

Areas of the catheter body having a greater degree of slits will be more flexible than those having lesser degrees of slits. In one embodiment, the slits can extend all the way through the elongate catheter. In other instances, some of the slits may be deeper or shallower than others which also affects the flexibility of the corresponding region. In some variations of the curved portion, a range of deflection between the flexible segments may be achieved. This may be accomplished through different geometric patterns of slits, different spacing of the slits, frequency of the slits, size of the slits, and so forth. In some instances, the degree of stiffness may be adjusted by adding additional spines of various lengths in certain areas or adjusting the width of the spines.

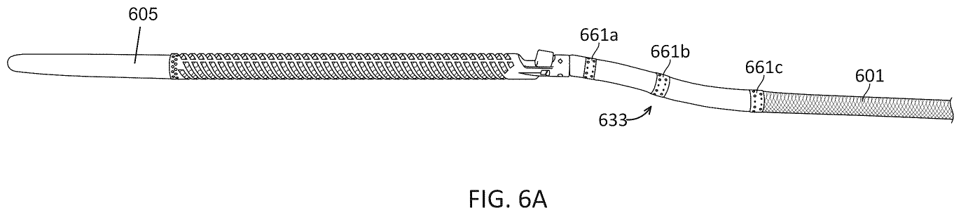

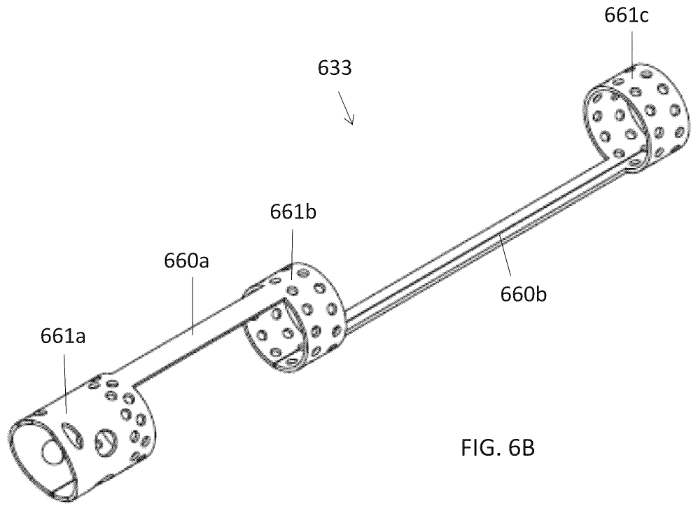

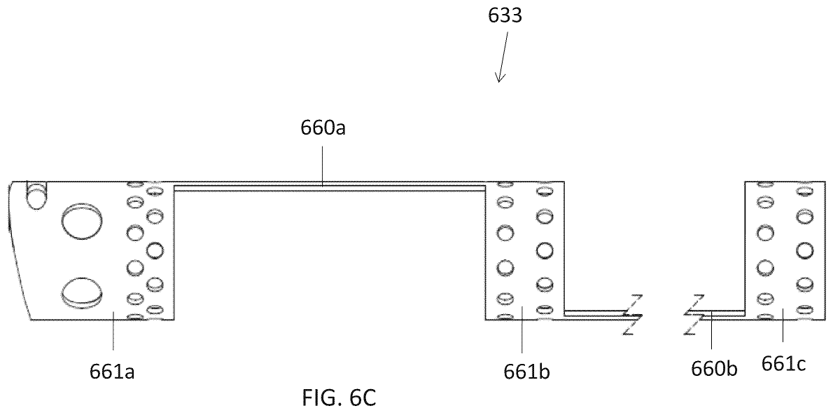

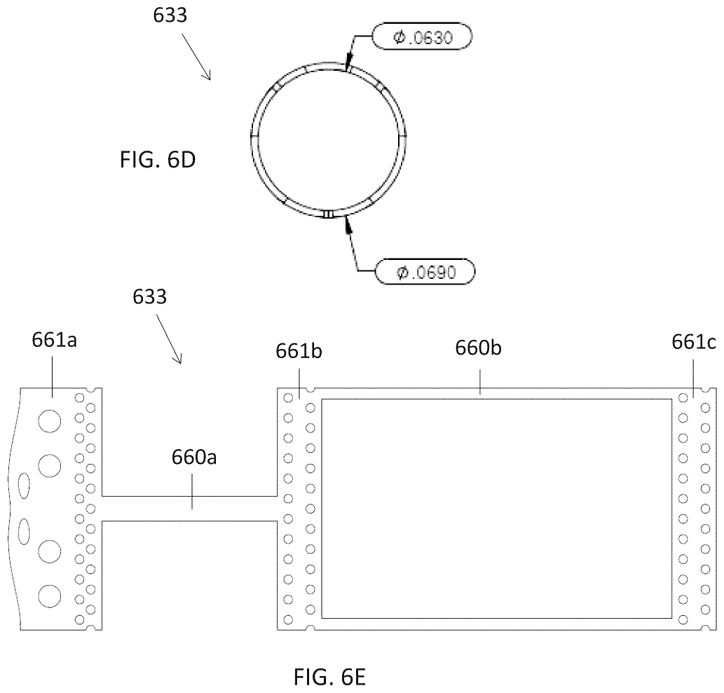

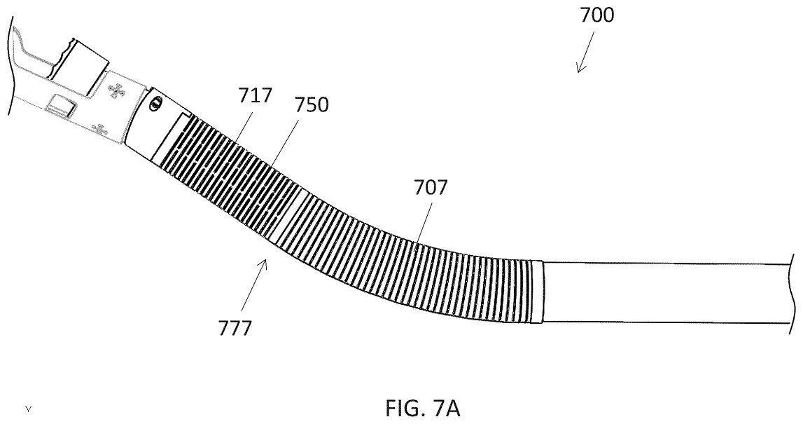

Referring to FIGS. 6A-6E, another exemplary curved portion 633 (e.g., for use as curved portion 433) is shown. The curved portion 633 includes a frame having three annular ring spines 661a,b,c connected together by two longitudinal spines 660a,b. The longitudinal spines 661a,b,c can be approximately 180° away from one another. In some embodiments, the distal ring 661a,bc can be beveled at the distal end, as shown in FIG. 6C, to allow for dropping or pivoting of the nosecone 605. Further, the space between the annular ring spines 661a,b,c and the longitudinal spines 660a,b can be open or cut-away (i.e., not include the frame material). In some embodiments, the frame can be laminated to the elongate body 601 with one or more thin polymer layers, such as Tecothane. The ring 661a,b,c can include holes therein for soldering or laminating the mechanism 633 to the elongate body of the catheter. In other embodiments, the frame can remain unlaminated to provide for greater flexibility. When compression is placed upon the mechanism 633, the mechanism 633 can bend away from each of the spines 633, forming an s-shape.

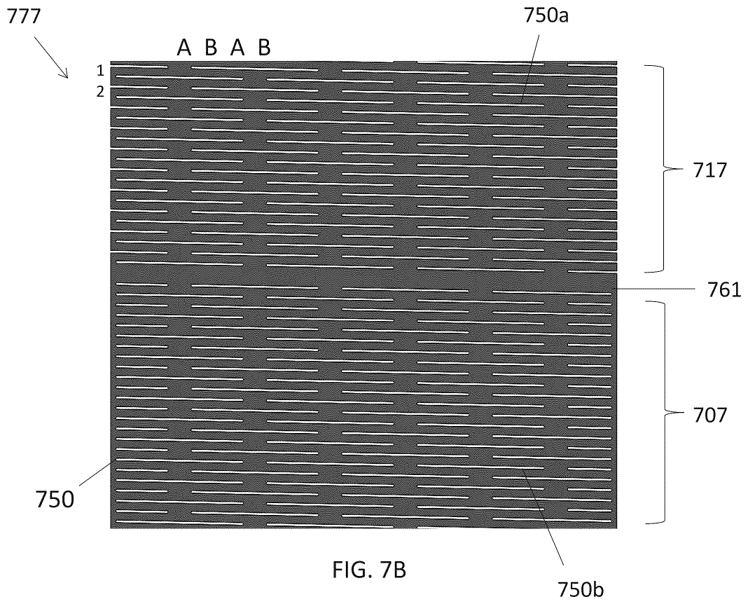

Referring to FIGS. 7A and 7B, in some embodiments, an atherectomy catheter 700 can include a curved portion 777 that includes a fixed jog section 707 and a flexible section 717. The fixed jog section 707 can either be proximal to the flexible section 717 (as shown) or distal to the flexible section 717. In some embodiments, the fixed jog section 707 is longer than the flexible section 717. For example, the fixed jog section 707 can be 5-10 mm, such as 8 mm, and the flexible section 717 can be 2-6 mm, such as 5 mm. Further, in some embodiments (as shown), the fixed jog section 707 can include only a single curve rather than a double curve (e.g., forming a c-shape rather than an s-shape). The angle of the curve can be, for example, 120° to 175°, such as 130° to 160°, such as approximately 145°. The flexible section 717 can be configured to bend passively during use (i.e., when acted upon by the vessel wall), for example to form an angle of between 90° and 180°, such as 110-170°, such as 130°-160°.

In some embodiments, the curved portion 777 can be made of a laminated frame. Referring to FIG. 7B, the curved portion 777 can include a frame that includes a plurality of circumferential slits 750a,b extending therethrough. The slits 750a of the flexible section 717 can extend entirely around the circumference (i.e., include no longitudinal spine therein) while the slits 750b of the fixed jog section 707 can end at a longitudinal spine 760 extending through the fixed jog section 707. An annular spine 761 can separate the flexible section 717 and the fixed jog section 707. The frame can be made, for example, of Nitinol or stainless steel. Further, the frame can be laminated with a thin layer of polymer, such as Tecothane, on one or both sides. In some embodiments, only the fixed jog section 707 is laminated while the flexible section 717 remains unlaminated.

Referring to FIG. 7B, the slits 750a,b can be arranged in a pattern that is configured to provide flexibility in the flexible section 717 while maintaining structural integrity of the elongate body in both the flexible section 717 and the fixed jog section 707. Thus, the majority of the slits 750a,b can have the same length, but be offset from one another. For example, the slits 750a in the flexible section 717 can be arranged in rows (1,2) and columns (A, B). Each slit 750a can have a length equivalent to the width of columns A+B+A. Further, the slits can be offset from one another by a distance of A+B. Thus, each column A can include slits from every row 1,2 while column B can include alternating slits (from either row 1 or 2). Column B thus provides structural integrity to the slitted portion of the device. The slits 750a of the flexible section 717 can provide flexibility to allow the catheter 700 to achieve the desired curvature in any direction when inside the body (i.e., the slits can pull apart on the outside of the curve and compress and/or overlap when on the inside of the curve). For example, the flexible section 717 can bend to align the cutter with the edge of the vessel.

Further, the slits 750b in fixed jog section 707 (except the shorter slits bordering the spine 560a) can likewise have a length equivalent to the width of columns A+B+A. Further, the slits can be offset from one another by a distance of A+B. Thus, each column A can include slits from every row 1,2 while column B can include alternating slits (from either row 1 or 2). In fixed jog section 707, however, the spine 760 can be heat-set to set the angle of the jog, fixing the jog.

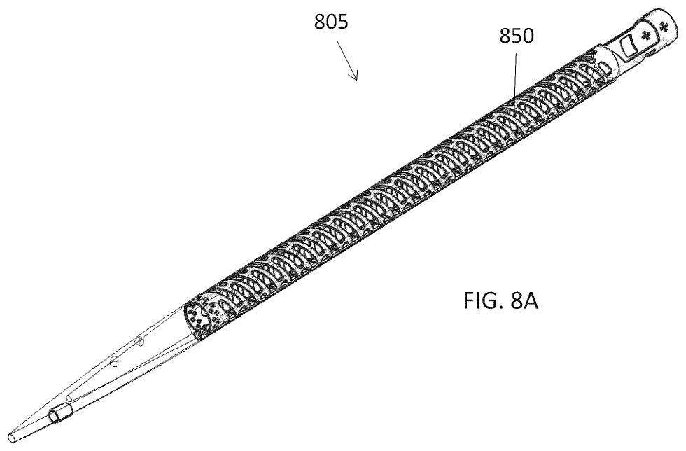

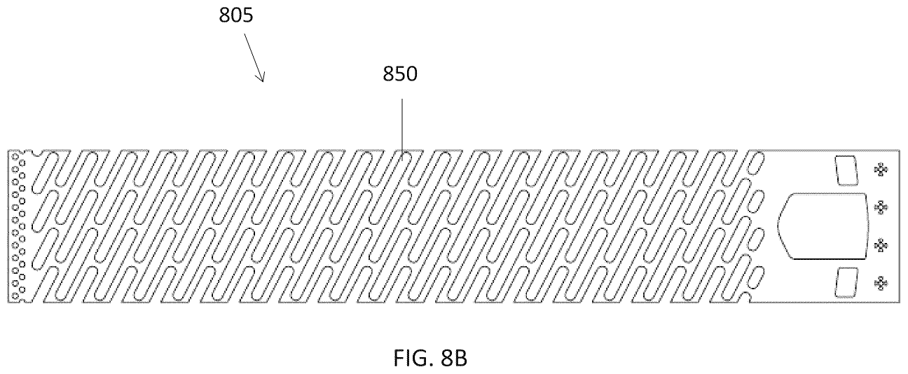

Referring to FIG. 8A, in some embodiments, the nosecone 805 can be flexible. That is, the elongate body can include one or more curves (as described herein), and the nosecone 805 can provide additional flexibility to allow the catheter to take the desired shape. The nosecone 805 can, for example, include a repeating laser cut pattern covered in a laminate layer. As shown in FIG. 8A, the pattern can include a series of spiral slits 850 extending around the circumference of the nosecone. some embodiments, the laser cut pattern can be cut out of stainless steal, which can be laminated with a polymer, such as Tecothane. Additional flexible nosecone designs are described in U.S. patent application Ser. No. 14/776,749, filed Sep. 15, 2015 titled “TISSUE COLLECTION DEVICE FOR CATHETER,” now U.S. Patent Application Publication No. 2016-0008025-A1 and International Patent Application No. PCT/US2017/035510, filed Jun. 1, 2017 and titled “CATHETER DEVICE WITH DETACHABLE DISTAL END,” both of which are incorporated by reference herein in their entireties. The flexible nosecone can be used in addition to, or in place of, any feature of the elongate body curved portions described herein.

In some embodiments, the curved portions of the elongate catheter bodies described herein can form a substantially s-shape with two different inflection points of opposite curvatures. In other embodiments, the curved portion can include a single inflection point that forms a substantially C-shape. Further, in some embodiments, one or more of the curves can be fixed. In other embodiments, one or more of the curves can be user activated (e.g., by pulling on the driveshaft or a separate pullshaft or wire). Further, any of the designs described herein can include a flexible section (e.g., of the elongate body or the nosecone) that allows the catheter to take the desired curvature during use.

In some embodiments, the amount of curvature of the user-adjusted curved portions can be further adjusted either prior to or during an atherectomy procedure based on the curvatures of the artery and the location of the plaque formation. For example, by tensioning a shaft of the catheter, the curved portion can constrict and adopt a sharper angle. Alternatively, when the shaft is relaxed, the curved portion can relax and adopt a wider angle. In such examples, the angles of deflection may be adjusted, for example, by 5 to 20 degrees.

In some embodiments, the user-adjusted curved portions can have a pre-shaped bend or curvature that can be further adjusted prior to or during an atherectomy procedure. In other embodiments, the curved portions can be straight before the user-activated bend is activated.

In any of the embodiments described herein, the nosecone can be configured to hold tissue that is debulked by the cutter. Further, the driveshaft and cutter can be configured to move distally to pack tissue into the nosecone.

In some embodiments, lamination of a framework can cause the laminating material to heat and shrink, pushing into open slits and fixing the shape of the frame (e.g., in a pre-shaped jog). For example, the curved portions 533 and/or 633 can be laminated so as to create a fixed jog that can either be further adjusted by pulling on the driveshaft or that remains fixed throughout the procedure. In other embodiments, lamination of the framework can keep the slits open and free of material, allowing for greater flexibility.

Any of the curved portions described herein may be used alone or in combination with a mechanism to deflect the nosecone. In some embodiments, the nosecone can be deflected by pulling on a cutter driveshaft. Such deflection mechanisms are described in U.S. patent application Ser. No. 15/072,272, filed Mar. 16, 2016, titled “ATHERECTOMY CATHETERS DEVICES HAVING MULTI-CHANNEL BUSHINGS,” now U.S. Pat. No. 9,592,075, and U.S. patent application Ser. No. 15/076,568 filed Mar. 21, 2016, titled “ATHERECTOMY CATHETERS AND OCCLUSION CROSSING DEVICES,” now U.S. Pat. No. 9,498,247, both of which are incorporated by reference in their entireties. In some embodiments, placing further tension on the drive shaft (i.e., after exposing the nosecone) can result in compression being applied to the curved portion, causing the curved portion to assume its final curved configuration. Having both the nosecone deflect and the curved portion can result in better tissue invagination and thus better or more efficient tissue cutting.

In embodiments where the nosecone is not deflected, the respective cutting windows can be optimized so as to allow for automatic invagination of tissue into the cutting window. Further, having the nosecone not deflect and relying entirely on the curved portion for tissue apposition can advantageously prevent the cutter from escaping from the nosecone during packing. Further, having the curved portion alone (i.e., without the nosecone activation) can advantageously eliminate having to use additional mechanisms to force a jog mid-surgery, such as pulling or pushing on a shaft, thereby enhancing both ease of use and enhancing image stability.

The atherectomy catheters having a curved portion described herein advantageously allows easier and closer positioning of the atherectomy cutter to plaque close to the inner artery walls. That is, the curved portions can be configured such that the exposed portion of the cutter (e.g., the area extending through the cutter window) moves closer to the vessel wall than the unexposed side of the cutter. This positioning can make cutting during the atherectomy procedure more efficient.

When a feature or clement is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An atherectomy device comprising:
a catheter body including:
a fixed jog section having a fixed curvature; and
a flexible section having a greater lateral flexibility than the fixed jog section, wherein the flexible section includes circumferential slits that are configured to allow the flexible section to passively bend in multiple directions;
a nosecone attached to a distal end of the catheter body; and
a driveshaft configured to rotate within the catheter body and to move distally and proximally within the catheter body, wherein a distal end of the driveshaft includes an annular cutter.

2. The atherectomy device of claim 1, wherein the circumferential slits are arranged in rows in a circumferentially offset pattern.

3. The atherectomy device of claim 1, wherein the fixed jog section is adjacent to the flexible section.

4. The atherectomy device of claim 1, wherein the fixed jog section includes a second set of circumferential slits.

5. The atherectomy device of claim 1, wherein the fixed jog section includes a longitudinal spine.

6. The atherectomy device of claim 1, wherein the catheter body further comprises a cutting window arranged to allow the annular cutter to extend therethrough.

7. The atherectomy device of claim 1, wherein the fixed jog section form an angle ranging from 120degrees to 175 degrees in the catheter body.

8. The atherectomy device of claim 1, wherein the catheter body further comprises an annular spine between the fixed jog section and the flexible section.

9. The atherectomy device of claim 1, wherein the fixed jog section has a c-shape.

10. The atherectomy device of claim 1, wherein the fixed jog section has an s-shape.

11. The atherectomy device of claim 1, wherein the nosecone is configured to pivot with respect to the catheter body.

12. The atherectomy device of claim 1, wherein the fixed jog section is proximal to the flexible section.

13. The atherectomy device of claim 1, wherein the fixed jog section is distal to the flexible section.

14. The atherectomy device of claim 1, wherein the fixed jog section and the flexible section are laminated in a polymer.

15. A method of performing atherectomy comprising:
inserting an atherectomy device into a vessel, the atherectomy device including:
a catheter body including:
a fixed jog section having a fixed curvature; and
a flexible section having a greater lateral flexibility than the fixed jog section, wherein the flexible section includes circumferential slits that allow the flexible section to passively bend in multiple directions within the vessel;
a nosecone attached to a distal end of the catheter body; and
a driveshaft that is rotatable within the catheter body and movable distally and proximally within the catheter body, wherein a distal end of the driveshaft includes an annular cutter;
positioning the catheter body within the vessel such that the fixed jog section urges the annular cutter against a wall of the vessel; and
rotating the annular cutter against the wall of the vessel.

16. The method of claim 15, further comprising moving the annular cutter distally to pack tissue into the nosecone.

17. The method of claim 15, further comprising removing the catheter body from the vessel with tissue packed within the nosecone.

18. The method of claim 15, wherein the annular cutter is extended through a window of the catheter body when the annular cutter is rotated against the wall of the vessel.

19. The method of claim 15, wherein the flexible section passively bends to an angle ranging from 130degrees to 160 degrees.

20. The method of claim 15, wherein the fixed jog section is adjacent to the flexible section.
